(19) United States
(12) Patent Application Publication
(10) Pub. No.: US 2004/0034322 A1
(43) Pub. Date: Feb. 19, 2004

(54) Plunger engagement section for safety hypodermic syringe

(76) Inventor: Pi-Chang Lo, Taoyuan City (TW)

Correspondence Address: M.K. Meditech Co., Ltd., P.O. Box No. 6-57, Junghe, Taipei, 235, TW

(21) Appl. No.: 10/643,989

(22) Filed: Aug. 20, 2003

Related U.S. Application Data
(63) Continuation-in-part of application No. 09/993,572, filed on Nov. 27, 2001, now Pat. No. 6,488,657.

(51) Int. Cl. A61M 5/00

(52) U.S. Cl. 604/181

(57) ABSTRACT

A plunger engagement section for safety hypodermic syringe includes a protruding portion at the plunger engagement section accommodated by a barrel. The protruding portion fills an entire space at an interior of a needle holder when the plunger is pushed forward, thereby overcoming a drawback or prior inventions as failing to completely inject a medical solute contained.

### RELATED APPLICATION

This application is a continuation-in part of U.S. application Ser. No. 09/993,572, filed on Nov. 27, 2001 and currently patented.

### BACKGROUND OF THE INVENTION

(a) Field of the Invention

The invention relates to a plunger engagement section for safety hypodermic syringe, and more particularly, to a plunger engagement section for safety hypodermic syringe that provides a novel structure in conjunction with the application Ser. No. 09/993,572.

(b) Description of the Prior Art

According to the aforesaid prior invention disclosed, a safety hypodermic syringe comprises a barrel, a needle holder, and a locating ring. The needle holder has a front engagement section press-fitted into the inside of a front small inner diameter section of the barrel, and a springy rear expansion section. The expansion section has a front side edge corresponding to a rear side of a shoulder of the barrel, and a rear side edge stopped at a protruding inner edge of a large inner diameter section of the barrel. The locating ring has an outside wall with an outer diameter slightly larger than a neck at the inner diameter section of the barrel. When the neck is inserted into the needle holder, the expansion section of the sleeve is expanded to butt at the large inner diameter section of the barrel, and therefore locating effects are provided by expanding the rear edge of the expansion section at the protruding edge. Hence, such type of safety hypodermic syringe adopts a locating mechanism formed by the needle holder, the locating ring and the barrel, so as to locate the needle holder at an end portion of the barrel and to perform an injection after placing in a needle.

For that a hollow chamber of the barrel is disposed with a needle holder, the needle holder is necessarily provided with a hollow tubular section for connecting to the needle. However, a space in an interior of the tubular section is prone to residual dosage and hence wastage of a portion of the medical dosage contained in the hollow tubular section. Furthermore, for barrels having smaller capacities, errors occurred by the hollow tubular section are likely to result in after-effects of insufficient dosages.

### SUMMARY OF THE INVENTION

The primary object of the invention is to provide a plunger engagement section for safety hypodermic syringe, so as to thoroughly inject a medical solute contained at an interior of a hollow tubular section of a needle holder thereof, and to further overcome drawbacks as failures of completely injecting medical solutes or uneven dosage concentrations.

To accomplish the aforesaid object, the plunger engagement section for safety hypodermic syringe comprises a protruding portion at a front end of the plunger engagement section accommodated by a barrel. In order to thoroughly inject a medical solute contained in the barrel, the protruding portion fills an entire space within a hollow tubular section at an interior of a needle holder of the syringe during injection, thereby overcoming a drawback as failing to completely inject the medical solute.

### BRIEF DESCRIPTION OF THE DRAWINGS

FIG. 1 shows a conventional exploded elevational view illustrating a safety hypodermic syringe of a prior invention.

FIG. 2 shows an exploded elevational sectional view illustrating the locating ring, the needle holder and the barrel of the prior invention.

FIG. 3 shows a sectional view illustrating the locating ring, the needle holder and the barrel of the prior invention.

FIG. 4 shows an exploded elevational view according to the invention.

FIG. 5 shows a sectional schematic view according to the invention.

FIG. 6 shows an operational schematic view according to the invention.

### DETAILED DESCRIPTION OF THE PREFERRED EMBODIMENTS

To better understand the technical contents of the invention, detailed descriptions shall be given with the accompanying drawings hereunder.

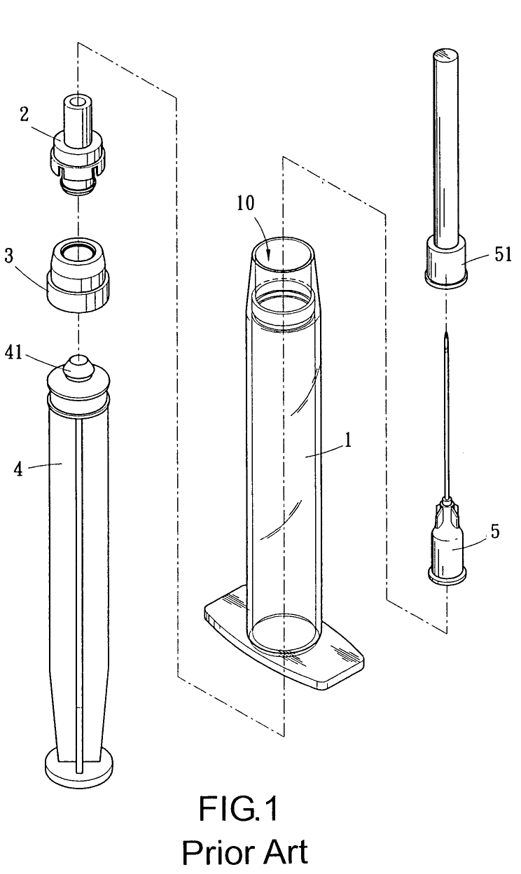

Referring to FIG. 1 showing an exploded elevational view of the prior invention, wherein the safety hypodermic syringe comprises a barrel 1, a needle holder 2, a locating ring 3, a plunger 4, a needle 5, and a needle shield 51.

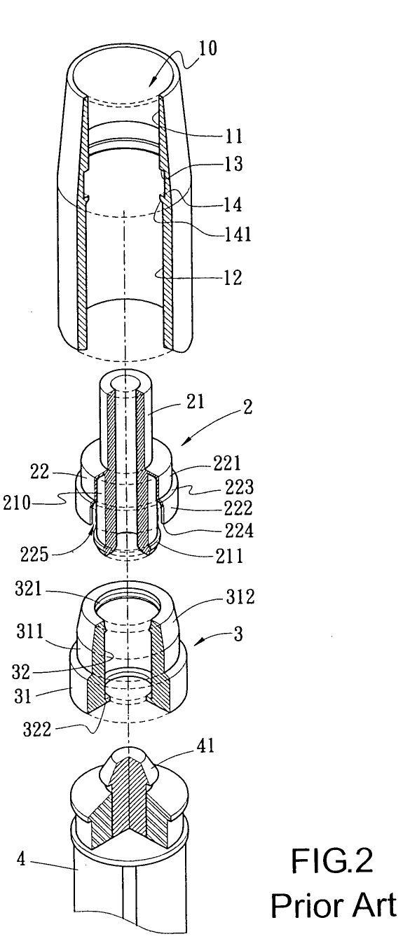

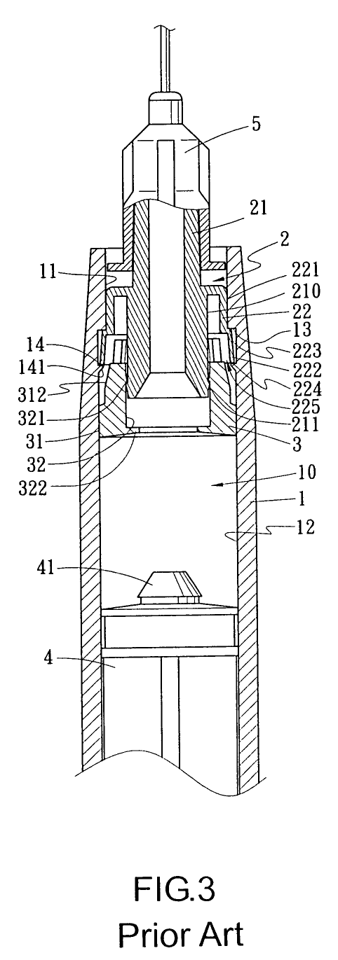

FIGS. 2 and 3 show an exploded elevational view and a sectional view of unassembled structures of the locating ring 3 and the needle holder 2.

The barrel 1 is a hollow tubular section having a hollow chamber 10 at an interior thereof. The hollow chamber 10 is has a front portion thereof as a small inner diameter section 11, and a rear portion thereof as a large inner diameter section 12. Between the small inner diameter section 11 and the large diameter section 12 is a shoulder 13. At an appropriate position approaching the shoulder 13, the large inner diameter section 12 has a flange 14 in a complete ring shape. The flange 14 is provided with a wedge-shaped rear beveled plane 141 for facilitating passing through of the needle holder 2.

A middle portion of an outer wall 210 of a hollow tubular section 21 of the needle holder 2 is disposed with a coaxial outer ring 22 in an outward direction. The coaxial outer ring 22 has a joining section 221 at a front portion thereof and an expansion section 222 at a rear portion thereof. The joining section 221 is for locating purposes when inserted into the small inner diameter section 11 of the barrel 1. The expansion section 222 has a front edge 223 and a rear edge 224, with the front edge 223 stopped at the shoulder 13 of the barrel 1 for locating purposes and the rear edge 224 corresponded at the flange 14 at the large inner diameter section 12 of the barrel 1. In addition, between the expansion section 222 and the outer wall 210 of the hollow tubular section 21 is an in-ring space 225.

The locating ring 3 has an outer wall 31 and an inner wall 32. The outer wall 31 is disposed to slide within the large diameter section 12 of the barrel 1. The inner wall 32 is disposed to slide around the outer wall 210 of the hollow tubular section 21 of the needle holder 2 as shown in FIG. 3. The outer wall is 31 is formed with a neck portion 311 at a front end thereof, and the neck portion 311 is further formed with a beveled plane at a front end thereof for favoring inserting into the in-ring space 225 of the needle holder 2, and has an outer diameter slightly larger than the inner diameter of the expansion section 222 of the needle holder 2. The inner wall 32 has a first fastening portion 321 and a second fastening portion 322 at front and rear ends thereof, respectively. The first fastening portion 321 is for corresponding with a first fastening portion 211 at a rear end of the hollow tubular section 21 of the needle holder 2. The second fastening portion 322 is for coordinating with an engagement section 41 at the front end of the plunger 4. Wherein, the first fastening portion 211 is a complete ring provided at the outer wall 210 of the hollow tubular section 21 in an outward direction, and the first fastening portion 321 is provided at the inner wall 32 of the locating ring 3 in an inward direction. Note that sections of the first fastening portions 211 and 321 are wedge-shaped in opposite directions for facilitating secure fastening as well as blocking disengagement.

The safety hypodermic syringe according to the invention utilizes a locating structure relationship of the needle holder 2 and the locating ring 3 that correspond with the flange 14 at the large inner diameter section 12 of the barrel 1, so as to locate the needle holder 2 at an end portion of the hollow chamber 10 of the barrel 1. Thus, an injection can be performed when the needle 5 is inserted and joined into the hollow tubular section 21 of the needle holder 2. However, the hollow chamber 20 within the hollow tubular section 21 is prone to residual dosage and hence wastage of a portion of a medical solute contained. Furthermore, for a barrel 1 having a smaller capacity, residual dosage incurs insufficient dosage and produces an error in the intended dosage.

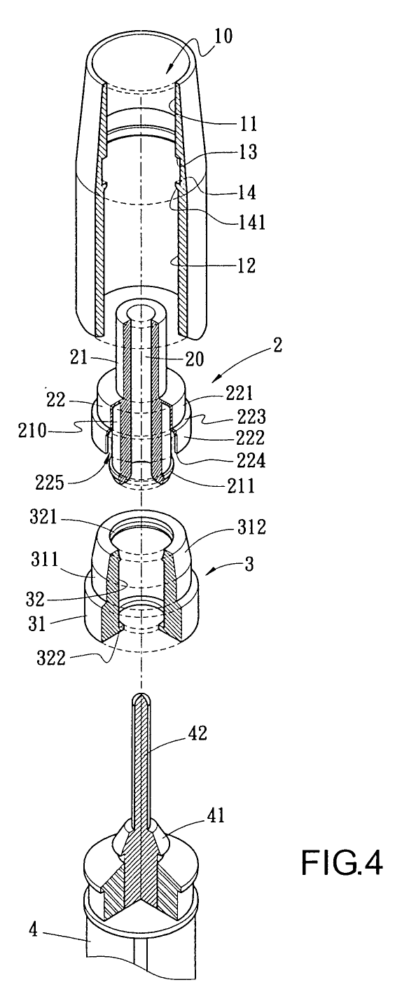

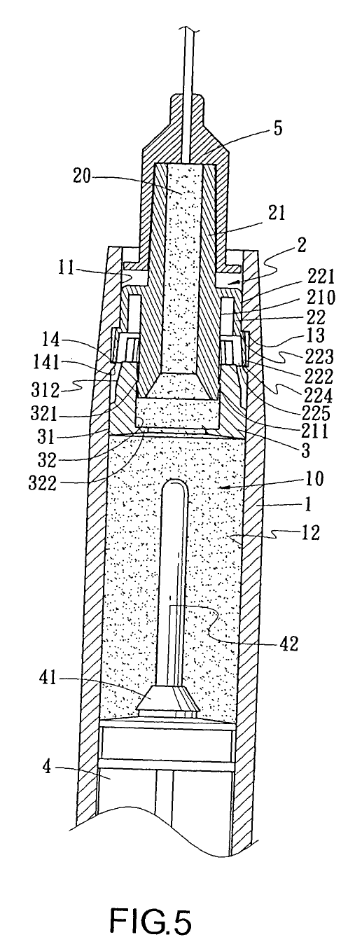

Therefore, referring to FIGS. 4 and 5 showing an exploded elevational view and a sectional schematic view according to the invention, to overcome the aforesaid drawback, the invention further comprises a protruding portion 42 at the engagement section 41 of the plunger 4. The protruding portion 42 is a cylindrical body having dimensions corresponding to the hollow chamber 20 within the hollow tubular section of the needle holder 2.

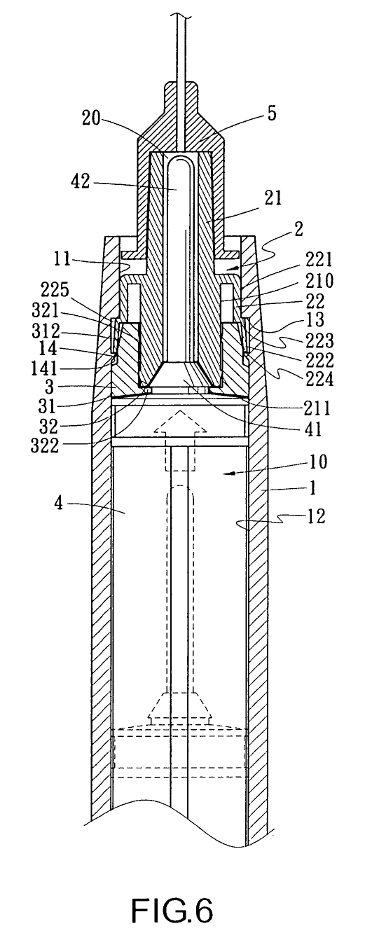

Referring to FIG. 6 showing a plunger according to the invention being pushed forward, when the plunger 4 is pushed forward to having completed injection, the engagement section 41 at the front end of the plunger 4 is extended into the locating ring 3 and corresponded with the second fastening portion 322. Meanwhile, the protruding portion 42 fills the space of the hollow chamber 20 within the hollow tubular section 21, thereby overcoming medical dosage wastage or uneven concentration of medical solutes by thoroughly injecting a medical solute contained in the barrel 1.

It is of course to be understood that the embodiment described herein is merely illustrative of the principles of the invention and that a wide variety of modifications thereto may be effected by persons skilled in the art without departing from the spirit and scope of the invention as set forth in the following claims.

What is claimed is:

1. A plunger engagement section for safety hypodermic syringe, applied in a safety hypodermic syringe having a barrel, a needle holder, a locating ring, a plunger, a needle and a needle shield, comprising the characteristics that:
the plunger accommodated in the barrel is provided with a protruding portion at a front end of the engagement section thereof, and the protruding portion fills a hollow space at an interior of the needle holder when pushing the plunger forward and performing an injection, thereby more thorough injecting a medical solute contained in the barrel.

2. The plunger engagement section for safety hypodermic syringe in accordance with claim 1, wherein the protruding portion is raised and extended from a middle portion of the engagement section for corresponding with the space at the interior of the needle holder.

3. The plunger engagement section for safety hypodermic syringe in accordance with claim 1, wherein the protruding portion is a cylindrical body having dimensions corresponding to a diameter of the space at the interior of the needle holder, thereby filling the space when pushing the plunger forward.
